(19) United States
(12) Patent Application Publication
(10) Pub. No.: US 2011/0036922 A1
(43) Pub. Date: Feb. 17, 2011

(54) Air-Freshener For a Vehicle

(75) Inventor: Dirk Jan Bulsink, Leiden (NL)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: SARA LEE, Utrecht (NL)

(21) Appl. No.: 11/579,897

(22) PCT Filed: Apr. 29, 2005
(86) PCT No.: PCT/NL2005/000325

§ 371 Date: Oct. 3, 2007

(30) Foreign Application Priority Data
May 10, 2004 (EP) 04076393.0

(51) Int. Cl. A61L 9/12

(52) U.S. Cl. 239/47; 239/58; 239/282; 239/74

(57) ABSTRACT

An air-freshener for a vehicle, in particular a motorcar, provided with a housing into which a container for a volatile liquid or a liquid containing one or more volatile components can be inserted, in the opening of which container a wick extending into the housing is fitted, and with a movable covering cap which, when the container with wick has been inserted into the housing, can cover the wick entirely or partially, and with a control member for setting the position of the covering cap. According to the invention, the wick is covered by a sideways movable covering cap, thereby making the air-freshener more compact.

The invention relates to an air-freshener for a vehicle, in particular a motorcar, provided with a housing into which a container foravolatile liquid or a liquid. containing one or more volatile components can be inserted, in the opening of which container a wick extending into the housing is fitted, and with a movable covering cap which, when the container with wick has been inserted into the housing, can cover the wick entirely or partially, and with a control member for setting the position of the covering cap.

EP1031446 discloses an air-freshener showing the features of the preamble, wherein a cap is axially moved along a wick thereby uncovering the wick. In this way, the wick is exposed and the wick, soaked by a fragrance contained in a bottle, can release the fragrance for providing a pleasant ambient atmosphere to the car and/or to expel odours that are undesired. Although the disclosed air-freshener is easily handed and small enough to attach in the car interior without problems, a desire exists to further minimize the disclosed apparatus.

Therefore, it is an object of the invention to provide a further minimized air-freshener, that provides a covering cap with adequate sealing of the wick in a covering position and that provides a proper and controllable distribution of fragrance in an uncovered position.

In an aspect, for attaining the above described objects, the invention provides an air freshener according to the features of claim 1.

By providing an air freshener, wherein the wick is covered by a sideways movable covering cap, dimensions of the air-freshener can be shortened considerably, since it is no longer necessary to provide room for elevating the covering member. By slight sideways movement, the wick is already uncovered and is able to distribute fragrance.

In a preferred embodiment, the covering cap comprises a first semi-open part that closes onto a second semi-open part. Further, preferably, a sliding mechanism is provided for slidingly moving the first part to and from said second part, wherein the control member comprises a knob for slidingly moving said first cap. In one embodiment, the second semi-open part may be fixed to the housing.

The housing may provided with ventilating openings via which, if a wick is arranged to extend in the housing, air can flow along the entirely or partially released wick. In an embodiment, wherein the first part is arranged to close the ventilation opening when covering the wick, vaporizing of the wick during covering is further minimized by providing a covering cap, wherein air circulation around the covering cap is minimized. Further ventilating openings may be provided in the wall of the housing.

In a further preferred embodiment, a fastening element may be present for securing the air-freshener on a strip-shaped element, in particular that of a guard of the fan in the dashboard of a motorcar. When the air-freshener is connected to the fan of the vehicle, the air flow through the air-freshener may be controllable both by setting the position of the covering cap, and by switching the fan of the vehicle faster or slower and/or by switching the temperature of the air flow in the vehicle higher or lower. In addition the housing may comprise a holding element for retaining an insertable and removable container for a volatile liquid or a liquid having one or more volatile components. Said holding element may be a flexible lip.

The invention is further related to a container for a volatile liquid or a liquid having one or more volatile components, in the opening of which a wick is insertable, said container having a shape that matches the air-freshener according to any one of the preceding aspects. An other aspect of the invention is a container for a volatile liquid or a liquid containing one or more volatile components for application in the air freshener. According to the invention, the container comprises a wick extending from an opening of said container a detachable covering cap for covering the wick wherein said covering cap having a dimension smaller than the wick so as to compress the wick while being covered. It is noted that this aspect can be protected separately from the inventive air-freshener.

Further features and benefits related to the invention are illustrated in the drawings. Herein:

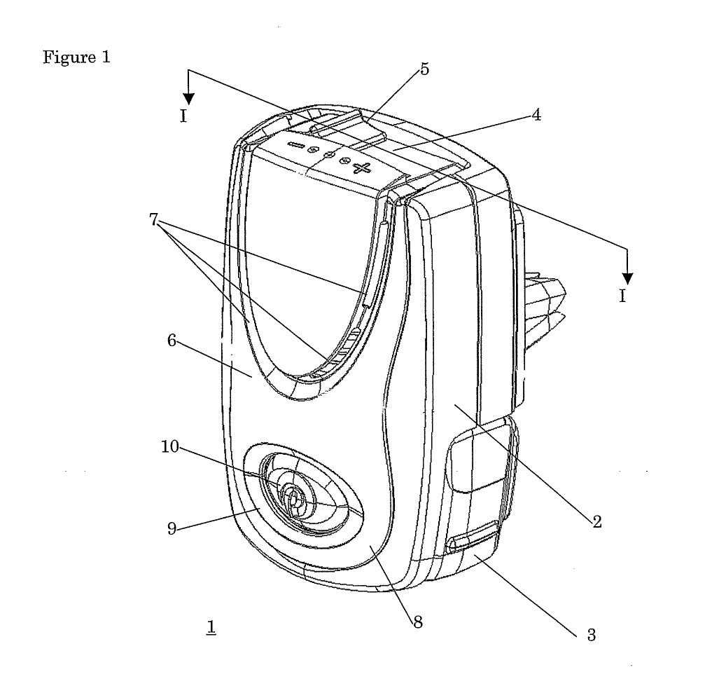

FIG. 1 shows a front perspective schematic view of an embodiment of the invention;

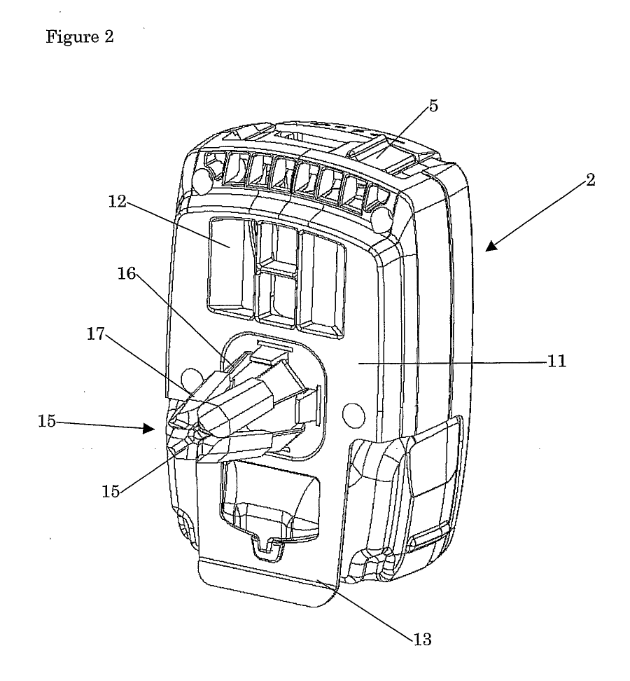

FIG. 2 shows a rear perspective schematic view of an embodiment of the invention;

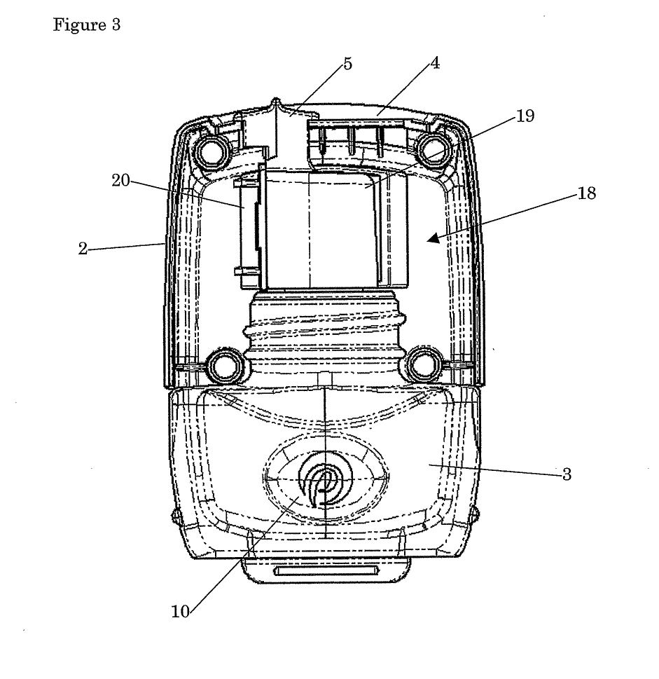

FIG. 3 shows a schematic sectional view along a line I-I in FIG. 1 of the covering cap in closed position;

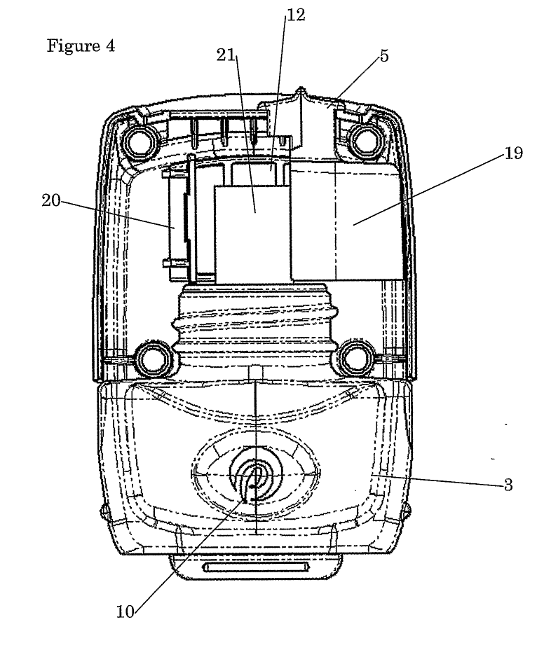

FIG. 4 shows a schematic sectional view along a line I-I in FIG. 1 of the covering cap in fully opened position; and

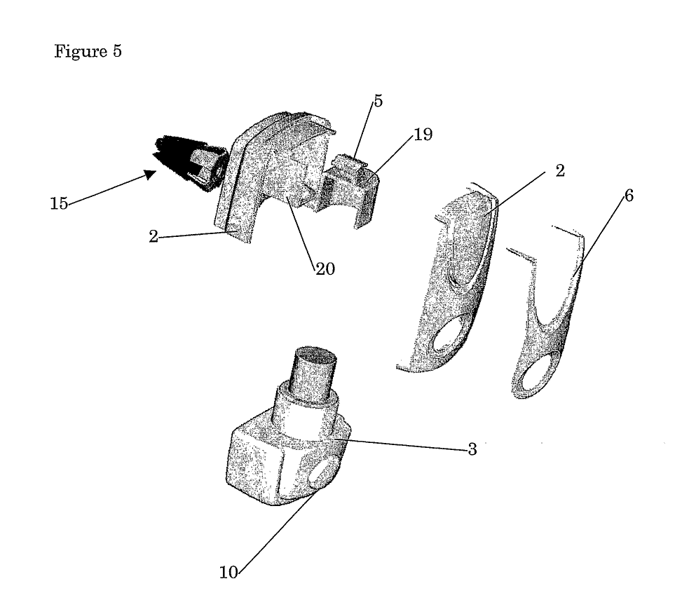

FIG. 5 schematically shows the embodiment of FIG. 1 in an exploded view.

In the figures, the same or corresponding items are referenced by the same reference numerals.

In FIG. 1 a front perspective schematic view of an embodiment of the air freshener according to the invention is illustrated. The air-freshener 1 is attachable to a site in the interior of a vehicle, in particular a motorcar, in a way further described with reference to FIG. 2. The air-freshener 1 is provided with a housing 2 into which a container 3 for a volatile liquid is inserted. A sliding mechanism 4 is present for slidingly moving a knob 5 that opens or closes a covering cap as will be further described with reference to FIG. 3. The air-freshener comprises an interchangeable front plate 6 that can be adapted to a custumers favourite colour or appearance. The front plate 6 comprises ventilation outlets 7 via which, when a wick is arranged to extend in the housing 2, air can flow along the entirely or partially released wick.

The front plate further comprises an extending lid 8 comprising an opening 9 that matches to a protrusion 10 that is provided on the container 3. The air outlets 7 are oriented partially sideways, so that fragrance is ventilated into a broad range of forward directions.

FIG. 2 shows a rear perspective schematic view of the air freshener illustrated in FIG. 1. The rear face 11 comprises a ventilation opening section 12, that allows air to be transported through the housing 2, so that air can flow along the entirely or partially released wick (not shown). The rear face is further provided with a flexible fixation clip 13 that snaps onto a protrusion that is provided on the container 3 on the rear side. In this way, the container is partially introduced into the housing 2, until snap fit of the clip 13. Furthermore, a fastening element 14 is present for securing the air-freshener on a strip-shaped element, in particular that of a guard of the fan in the dashboard of a motorcar. The fastening element 14 comprises four clamping elements 15 projecting outwards from the housing 2, which clamping elements 15 are separated from each other according to two planes which are at least substantially perpendicular to each other, to enable securing the air-freshener 1 either on a vertical, or on a horizontal strip-shaped element. The clamping elements 15 are formed by metal strips 16 provided, at least at the ends thereof, with a plastic covering layer 17.

FIG. 3 shows a schematic sectional view along a line I-I in FIG. 1. In FIG. 3, the covering cap 18 is illustrated in closed position. Here the wick (see FIG. 4) is completely covered by the covering cap 18, so that the wick can not vaporize fragrances contained in the container 3. The covering cap 18 comprises a first semi-open part 19 that closes onto a second semi-open part 20. The second semi-open part 20 is fixed to the housing 2. First cover part 19 is connected to a knob 5 that can move along a sliding mechanism 4. Through this sliding movement the cover part 19 is sideways movable from the wick, so as to uncover said wick as is illustrated in FIG. 4.

FIG. 4 shows the embodiment of FIG. 3 in fully opened position of the covering cap 18. The wick 21 is now uncovered and ventilation is provided via the ventilation openings 12 along the entirely or partially released wick 23. By arranging the covering cap 18 according to the invention, the apparatus can effectively be dimensioned according to the dimension of the container 2 comprising the wick 23, and no extra latitude is needed for providing axial movement to the cap 18.

FIG. 5 schematically shows the embodiment of FIG. 1 in an exploded view. In use, the rear part 22 and front part 23 of the housing are connected. The front plate 6 however can be clipped interchangeably to the front part 25 so that the user can modify the appearance of the air-freshener to correspond to his particular preference. Furthermore, the container 2 comprising the wick 23 can be of a refill type or of a replacement type and is specifically adapted to match the clip element 13 described with reference to FIG. 2. The front plate 6 comprises a see-through opening for identifying the fragrance level in the container.

Although in the drawings the covering cap comprises a fixed part and a slidable part, other embodiments may comprises a covering cap that slides or splits the covering cap. Such variations are deemed to fall within the scope of the attached claims.

1. An air-freshener for a vehicle, comprising:
a housing;
an insertable and removable container constructed and arranged to be placed in the housing and adapted to contain a volatile liquid or a liquid containing one or more volatile components;
a wick extending from an opening of the container;
a laterally movable covering cap constructed and adapted to cover the wick when the container is inserted into the housing;
a fastening element for securing the air-freshener on a guard of a fan in a dashboard of the vehicle;
and a control member for setting the lateral position of the covering cap with respect to the wick, wherein the wick may be at least partially uncovered by the position of the covering cap.

2. The air-freshener according to claim 1, wherein the covering cap comprises a first semi-open part that closes onto a second semi-open part.

3. The air-freshener according to claim 2, further comprising a sliding mechanism for slidably moving the first part to and from said second part, the control member comprising a knob for slidably moving said first cap.

4. The air-freshener according to claim 2, wherein the second semi-open part is fixed to the housing.

5. The air-freshener according to claim 2, wherein the housing is provided with at least one ventilating opening, wherein air can flow along the at least partially uncovered wick.

6. The air-freshener according to claim 5, wherein the first part is adapted to at least partially close the at least one ventilating opening when covering the wick.

7. The air-freshener according to claim 5, wherein the at least one ventilating opening comprises a plurality of ventilating openings in at least one wall of the housing.

8. (canceled)

9. The air-freshener according to claim 8, wherein when the air-freshener is connected to the fan, air flow through the air-freshener may be controlled by: setting the position of the covering cap, adjusting the speed of the fan, adjusting the temperature of the air flow in the vehicle, or a combination thereof.

10. The air-freshener according to claim 1, wherein the housing comprises a holding element for retaining the container.

11. An air-freshener for a vehicle, comprising: a housing; an insertable and removable container constructed and arranged to be placed in the housing and adapted to contain a volatile liquid or a liquid containing one or more volatile components; a wick extending from an opening of the container; a laterally movable covering cap constructed and adapted to cover the wick when the container is inserted into the housing; and a control member for setting the lateral position of the covering cap with respect to the wick, wherein the housing further comprises a face plate provided with a transparent opening for identifying a liquid level in the container, and wherein the wick may be at least partially uncovered by the position of the covering cap.

12. A container for a volatile liquid or a liquid having one or more volatile components, comprising: a wick extending from an opening of the container; and a detachable cover for covering the wick while not inserted in the air freshener, said cover having a dimension smaller than the wick so as to compress the wick while being covered, wherein said container is adapted to be insertably and removably arranged in an air-freshener, comprising:
a housing adapted to receive the container;
a laterally movable covering cap constructed and adapted to cover the wick when the container is inserted into the housing; and
wherein the lateral position of the covering cap may be adjusted with respect to the wick, wherein the wick may be at least partially uncovered by the position of the covering cap.

13. (canceled)

14. The container according to claim 12, further comprising a transparent protrusion portion for identifying a liquid level in the container.

15. An air-freshener for a vehicle, comprising: a housing; an insertable and removable container constructed and arranged to be placed in the housing and adapted to contain a volatile liquid or a liquid containing one or more volatile components; a wick extending from an opening of the container; a laterally movable covering cap constructed and adapted to cover the wick when the container is inserted into the housing; and a control member for setting the lateral position of the covering cap with respect to the wick, wherein the wick may be at least partially uncovered by the position of the covering cap, and wherein the container further comprises a detachable cover for covering the wick while not inserted into the air freshener, said covering cap having a dimension smaller than the wick so as to compress the wick while being covered.

16. A container for a volatile liquid or a liquid having one or more volatile components, comprising:
a wick extending from an opening of said container; and
a detachable covering cap for covering the wick, said covering cap having a dimension smaller than the wick so as to compress the wick while being covered.

17. The air-freshener according to claim 1, wherein the container comprises a transparent protrusion portion for identifying a liquid level in the container.

18. An air-freshener for a vehicle, comprising:
a housing comprising at least one ventilating opening;
an insertable and removable container constructed and arranged to be placed in the housing and adapted to contain a volatile liquid or a liquid containing one or more volatile components;
a wick extending from an opening of the container;
a laterally movable covering cap comprising a first semi-open part that slidably closes onto a second semi-open part so as to cover and uncover the wick when the container is inserted into the housing; and
a control member for setting the lateral position of the covering cap with respect to the wick, wherein the wick may be at least partially uncovered by the position of the covering cap,
wherein when the covering cap is in a closed position, air continues to flow through the ventilating openings.
